(19) United States
(12) Patent Application Publication
(10) Pub. No.: US 2010/0211006 A1
(43) Pub. Date: Aug. 19, 2010

(54) MEDICAL GUIDE WIRE TORQUING DEVICE AND METHOD OF APPLYING TORQUE USING THE DEVICE

(76) Inventor: Esko Jens Schmidt-Sørensen, Praesto (DK)

Correspondence Address: WINSTON & STRAWN LLP;PATENT DEPARTMENT, 1700 K STREET, N.W., WASHINGTON, DC, 20006, US

(21) Appl. No.: 12/738,563

(22) PCT Filed: Oct. 22, 2008
(86) PCT No.: PCT/EP08/64263

§ 371 Date: Apr. 21, 2010

(30) Foreign Application Priority Data
Oct. 22, 2007 (EP) 07118968.2

(51) Int. Cl. A61M 25/09

(52) U.S. Cl. 604/95.01

(57) ABSTRACT

A medical guide wire torquing device that includes an elongated body defining a longitudinal axis (A) and provided with an element for receiving the guide wire. The receiving element includes a through-going bore extending inside the elongated body and a clamping body for securing torque on the guide wire in response to application of an external force in a first position and for releasing the torque from the guide wire in a second position. The wall of the elongated body has a traversing opening, and an actuator is arranged in the traverse opening in contact with the clamping body for actuating the clamping body between the first and second positions. The torque is easy to manipulate using only one hand and can be placed lengthwise of the guide wire in alternate secured and released positions without kinking the guide wire.

The present invention relates to a medical guide wire torque comprising an elongated body defining a longitudinal axis and provided with means for receiving the guide wire, which means includes a through-going bore extending inside the elongated body and accommodating a clamping body for in response to application of an external force in a first position securing the torque on the guide wire and in a second position releasing the torque from the guide wire.

The invention also relates to a method of using the medical guide wire torque.

Diagnostic procedures, such as e.g. angiography and surgical procedures for placing endovascular devices, such as stents to obliterate aneurysms or alleviate occlusive diseases, involve the use of medical guide wires. Intubation of arteries or other kinds of vessels are however often difficult to carry out because the medical guide wire needs to be moved forth and back and rotated inside the vessel in order to reach the desired position without creating damages. Hence, a high degree and freedom of directional manipulation of the medical guide wire is very important to both surgeon and patient. Various kinds of medical guide wire torques, which the surgeon can manipulate using only one hand, are suggested in the art.

U.S. Pat. No. 6,030,349 discloses a medical guide wire torque composed of an elongated main body provided with a longitudinal, extending recess for receiving the guide wire. The main body has a vertically extending hole in which a button is displaceable located. When the button is depressed the part of the guide wire extending in the recess is sagged and displaced beyond the longitudinal axis of the main body to prevent lengthwise movement of the torque along the guide wire. Each time the button is depressed sharp edges and bends are made on the guide wire. As a result, the guide wire is permanently damaged which makes further advancement of the guide wire towards a target inside the vessel difficult, as well as the bends may damage the vessel wall.

Another torque for one-hand use is known from U.S. Pat. No. 5,392,778. This known torque consists of an elongated main body terminating in a set of flexible prongs. These prongs are received in a tubular body, which is arranged concentric around the main body at the guide wire exit end. The main body has an annular locking protrusion which engages a corresponding annular recess on the tubular body in which engaged position the prongs are spread apart. The forward longitudinal, axial displacement of the tubular body in relation to the main body disengages the engagement of the protrusion and the recess, and forces the prongs towards each other in a tweezers-like manner around the guide wire. This known device is highly sensible to unintentional action of external forces. If for example the tip of the tubular body hits something or is unintentionally touched by the surgeon, unintentional loosening of the tweezing force is likely to occur. Moreover, the contact area between the prongs and the guide wire is very small.

In a first aspect according to the present invention is provided a medical guide wire torque of the kind mentioned in the opening paragraph which facilitates selective placement on a guide wire in a feasible and effective way.

In a second aspect according to the present invention is provided a medical guide wire torque of the kind mentioned in the opening paragraph which is easy to torque using only one hand.

In a third aspect according to the present invention is provided a medical guide wire torque of the kind mentioned in the opening paragraph which does not damage the medical guide wire during use.

In a fourth aspect according to the present invention is provided a medical guide wire torque of the kind mentioned in the opening paragraph which can be used for medical guide wires of different diameters.

In a fifth aspect according to the present invention is provided a medical guide wire torque of the kind mentioned in the opening paragraph which is easy and inexpensive to manufacture.

The novel and unique features whereby this is achieved is the fact that the wall of the elongated body has a traversing opening, and an actuation means is arranged in the traverse opening in contact with the clamping body for actuating the clamping body between the first and second positions.

The actuation means serves advantageously for applying an external force at least transverse to the longitudinal axis to trigger the clamping body to act on the guide wire, to either hold on firmly to the guide wire or allow the guide wire to pass freely though the clamping body.

A firm hold of the medical guide wire torque on the guide wire is required when the surgeon needs to advance a distance of the guide wire further inside the vessel. In this first, secured position of the clamping body on the guide wire the torque also serves as a handy manipulation grip, which also allows controlled rotation of the guide wire and steering of the tip of the guide wire.

In the second, released position of the clamping body the medical guide wire torque is set free. The medical guide wire torque is moved backwards on the guide wire and repositioned at any suitable location, and the surgeon is able to advance a further length of the guide wire into the desired intubated vessel.

The torque advantageously serves for enlarging the diameter of the guide wire so that the surgeon has something to hold on when manipulating the guide wire.

If the through-going bore has a section of enlarged cross-sectional area and the clamping body is provided in said section, the clamping surfaces of the clamping body can be given a large contact area for the guide wire.

In the preferred embodiment of a medical guide wire torque according to the present invention the clamping body may be a clamping body comprising two opposed, elongated parallel wall parts diverging from each other towards the opening to define a gap for receiving the guide wire.

Due to the diverging wall parts the distance of the gap decreases in the direction away from the through-opening in the elongated body enabling the use of the medical guide wire torque on a wide range of guide wire diameters without compromising the clamping force. Hence, a very thin diameter guide wire may be located deeper in the gap than a thicker guide wire. The opposed, elongated parallel wall parts clamps the various sized guide wire in-between them in a safe and strong manner.

The opposed walls may or may not be interconnected opposite the traverse opening. In other words the clamping body may be constructed as a single unit consisting of two hinged wall parts or be arranged in the internal cavity as two single wall part units in close proximity opposite the traverse opening. Each embodiment has its advantages.

In case of a single unit the entire clamping body can be made of the same material and is very easy to manufacture and mount inside the elongated body. By choosing a material, which in itself is flexible, the clamping body can be designed with integrated flexible memory. When e.g. an external force is applied to the actuation means to displace said actuation means lengthwise of and at greater distance from the longitudinal axis in order to shift from the second position to the first position the clamping body automatically reassumes its initial shape and promptly takes a firm clamping grip on the guide wire. Moreover, the hinged relationship of the two wall parts can be utilized to prevent the walls from mutual axial displacement and/or offsetting in response to relocation of the medical guide wire torque on the guide wire.

In case the clamping body consists of two separate wall parts, the walls of the clamping body can be given different properties, by selective choice of material. As an example the wall can be made with different frictional gripping abilities.

In any of the embodiments the surfaces of the gap between the wall parts engaging the guide wire may be made with a pattern for improved engagement.

In an advantageous embodiment according to the present invention the medical guide wire torque further comprises a spring means for forcing the elongated walls of the clamping body towards each other to hold the guide wire firmly in the first position. Such a spring means may be provided whether or not the wall parts of the clamping body are connected to each other.

Preferably the spring means may be a substantially U-shaped spring, which engages at least the part of the clamping body opposite the traverse opening.

Alternative the spring means may be an integrated part of the clamping body, for example by embedding the spring means inside a clamping body having interconnected wall part with no memory shape.

When the actuation means is an actuation button extending through the traverse opening an external force can be transferred to the clamping body in a particular easy way.

The actuation means may expediently be sized to allow said means to be displaceable in at least one of the directions substantially along the longitudinal axis of the elongated body and substantially perpendicular to the longitudinal axis of the elongated body. When the actuation means is depressed the gap between the opposing walls is forced open, the clamping body is in its second position, and the guide wire is set free of the medical guide wire torque to move freely in the through-going bore. When the medical guide wire torque subsequently needs to be secured onto the guide wire, to lock the guide wire inside the clamping body in the first position, the actuation means may be axially, lengthwise displaced which triggers the actuation means to move perpendicularly away from the longitudinal axis of the elongated body. A very good contact and grip between the fingers and the medical guide wire torque may be achieved if the gripping part has an uneven exterior surface.

At least the opposing faces of the opposed wall parts of the clamping body has a coefficient of friction of at least 0.02, preferably at least 0.03. It has been found that this value provides a frictional contact between the surface of the guide wire and the clamping body which allows unobstructed intentional movement of the torque along the length of the guide wire in the second position of the clamping body, and a firm engagement between the guide wire and the clamping body when the clamping body is in the first position, which engagement allow for safe maneuvering and location of the guide wire inside the vessel.

A method of using the medical guide wire torque is also disclosed, which method comprises the steps of positioning a guide wire in the through-going bore of the elongated body, and activating the actuation means to control the clamping force of the clamping body on the guide wire. The method further comprises any of the steps of displacing the torque in the second position of the clamping body and rotating the torque fixed on the guide wire in the first position of the clamping body. These further steps of displacing and/or rotating the clamped guide wire expediently accomplish maneuvering of the guide wire inside the vessel.

The invention will described in further details below with reference to the accompanying drawing in which

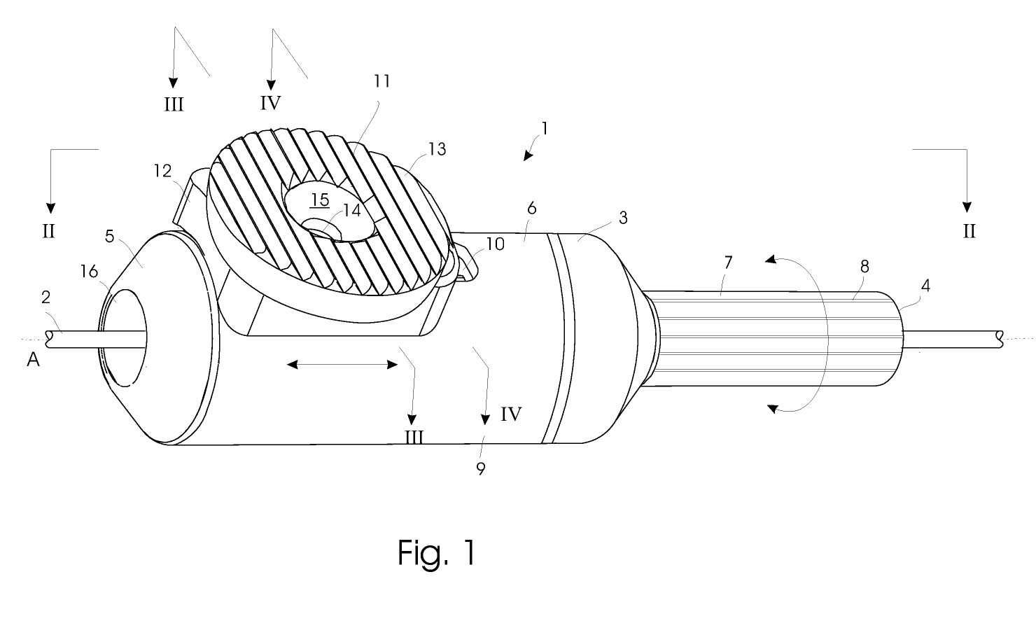

FIG. 1 shows a perspective view of a first embodiment of a medical guide wire torque according to the present invention,

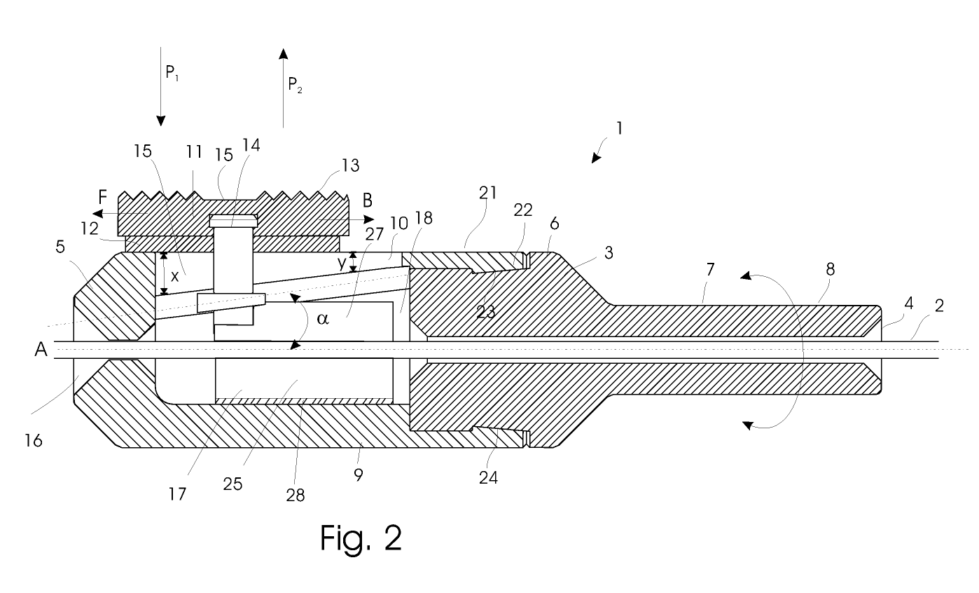

FIG. 2 shows in part, a longitudinal sectional view taken along the line II-II in FIG. 1,

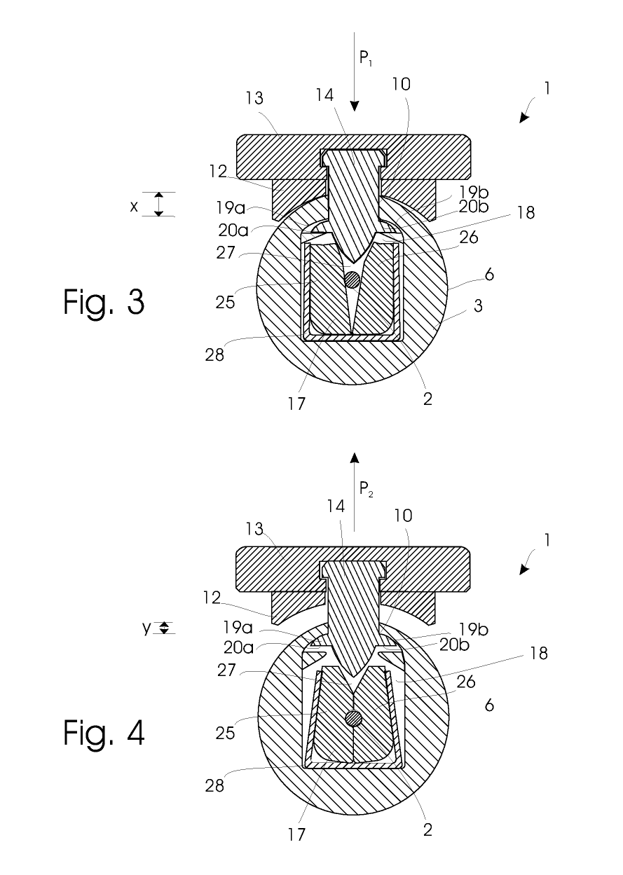

FIG. 3 shows a cross-sectional view taken along line III-III in FIG. 1 wherein the clamping body is in its first position, and

FIG. 4 shows a cross-sectional view taken along line IV-IV in FIG. 1 wherein the clamping body is in its second position.

The dimensions and geometrical shape of the medical guide wire torque are in the figures shown by way of example. Within the scope of the present invention the elongated body and the actuation means may be given any suitable exterior design.

FIG. 1 shows the medical guide wire torque 1 mounted on a guide wire 2.

The torque 1 is composed of an elongated body 3 having an inlet end 4 for a guide wire 2 and an opposing exit end 5 for the guide wire. The elongated body 3 has a main body part 6, which extends into a gripping part 7 of reduced cross-section. The gripping part 7 is provided with an uneven surface in the form of circumferential spaced apart recesses 8. The recesses provide the exterior surface of the gripping part 7 with a knurled surface that enhances gripping force when the torque 1 is to be rotated as indicated with the arched arrow around the gripping part. Any other kind of surface that improves gripping force is anticipated within the scope of the present invention. For example the surface may be provided with a silicone coating for enhancing frictional force between the fingers and the gripping part 7.

The main body part 6, which tapers towards the exit end 5, is defined by a circumferential wall 9 having a traverse opening through which the actuation means 11 extends, as will be explained in more detail with reference to FIG. 2.

The actuation means 11 includes a slider 12 provided with a fingerplate 13 and a wedge means 14. The fingerplate 13 has, in the embodiment shown, a cavity 15 for accommodating a fingertip so that at good grip of the slider can be obtained. The cavity is optional and other means that improves the degree of contact between the finger and the fingerplate 13 may be used.

A through-going bore 16 extends along the longitudinal axis A inside the elongated body 3.

FIG. 2 shows a sectional view taken along the longitudinal axis of the torque 1 shown in FIG. 1 with a guide wire 2 inserted.

The guide wire 2, the wedge means 14 and one wall part of the clamping body 17 are illustrated in full.

The traverse opening 10 opens into a lengthwise hollow space 18 inside the main body 6. This hollow space 18 has a cross-sectional area, which allows the clamping body 17 to be inserted in communication with the actuation means 11 through the traverse opening 10. The inside face of the wall 9 of the hollow space 18 has opposing guide means 19a,19b for engaging corresponding arms 20a,20b on the wedge means 14.

The elongated body 3 may be moulded or otherwise made as one single integrated part into which the clamping body 17 is inserted, e.g. via the traverse opening 10. Alternatively, the clamping body is inserted during the manufacturing process. In the embodiment shown in FIGS. 1 and 2 the annular end 21 of the main body 6 opposite the exit end 5 is provided with an internal circumferential protrusion 22 designed to snap fit into a corresponding external circumferential recess 23 on the end 24 of the gripping part 7 opposite the inlet end 4. In this embodiment the clamping body 17 is mounted inside the hollow space 18 before the gripping part is united with the main body 6.

The guide means 19 is in the embodiment shown in FIGS. 1 and 2 made as lengthwise, angled slots 19 along the interior face of the wall 9. The slots 19 are arranged in an acute angle α in relation to the longitudinal axis seen from the exit end 5. This means that the distance between the slot and the longitudinal axis at the exit end 5 is smaller than the distance at the annular end 21 of the main body 6.

Various alternative guide means can, within the scope of the present invention, be used instead of slots. The same effect can e.g. be obtained by adjusting the thickness of the wall 9 at the traverse opening so that the thickness is largest at the exit end 5. In the embodiment the actuation means may advantageously further comprise a compression spring inserted between the slide 12 and the wall 9, to assist the return movement of the wedge means after the wedge means has been depressed into the clamping body 17. In yet an alternative embodiment a slot-like alternative can be made by arranging an angled protruding rail along the interior wall of the bore.

Irrespective of which guide means 19 that are used these guide means provides for the actuation means 11, in particular the wedge means 14, to move along the longitudinal axis A of the elongated body 3 while at the same time varying the radial distance of the actuation means 11 to the longitudinal axis A. As a result of this design, moving of the actuation means towards the exit end 5, i.e. in the forward direction indicated with the arrow F, forces the wedge means 14 down into the gap 27 between the opposing wall parts 25,26 of the clamping body 17 to spread apart these wall parts 25,26 and open the gap 27 to set the guide wire 2 free of the clamping force exerted by the clamping body 17. By return movement of the actuation means 11 in the direction B, the wedge means 14 is moved away from the gap 27, which triggers the opposing wall parts 25,26 to move against each other and clamp around the guide wire 2.

These two situations are seen more clearly in FIGS. 3 and 4.

FIG. 3 shows a cross-sectional view taken along line III-III in FIG. 2 illustrating the actuation means 11 in the second position of the clamping body 17 in which the torque 1 is fixed and secured to the guide wire 2 in a non-displaceable manner so that the tip of the guide wire is allowed to be moved both lengthwise and rotational using only one hand to reach a target area inside a vessel.

FIG. 4 shows a cross-sectional view taken along line IV-IV in FIG. 2 illustrating the actuation means 11 in the first position of the clamping body 17 in which the torque 1 is not fixed and secured to the guide wire 2. In this first position of the clamping body 17 the torque 1 can be moved freely along the guide wire 2 using only one hand to reposition the torque 1 on the guide wire 2.

As is clear from FIGS. 3 and 4 a spring means 28 in the form of a U-shaped spring clamp 28 is arranged around the clamping body 17. This spring clamp 28 applies a spring force to the wall parts 25,26, which spring force holds the wall parts 25,26 in close proximity in the first position of the clamping body 17 where the wedge means 14 are out of the gap 27 between the wall parts 25,26, that is to say when the wedge means are moved or positioned in the direction indicated with the arrow P₂. When the wedge means are forced down, as indicated by the arrow P₁, into the gap between the wall parts 25,26 of the clamping body 17 the legs of the spring clamp are forced apart and tensioned to that once the wedge means 17 again are free of the clamping body the legs of the spring clamp returns to the position shown in FIG. 4, i.e. the first position of the clamping body 17.

The wedge means can be forced down into the gap 17 between the wall parts 25,25 be simply depressing the actuation means 11 in a direction P₁ perpendicular to the longitudinal axis A, using e.g. the thumb and holding the torque 1 in the same hand. Hence, lengthwise movement is not always required for shifting from the first position to the second position of the clamping body.

The torque according to the present invention is operated different than known torques in which depression of a button locks the guide wire inside the device.

The medical guide wire torque according to the present invention and the operating principle and technique constitutes a preferred alternative to known torques. The novel torque is very easy to use using only one hand; it is reliable and does not kink the guide wire.

1.-14. (canceled)

15. A medical guide wire torque device comprising:
an elongated body defining a longitudinal axis (A) and having a wall that includes a traversing opening;
a clamping body;
means for receiving a guide wire, the receiving means including a through-going bore extending inside the elongated body and accommodating the clamping body for, in a first position, securing the torque on the guide wire in response to application of an external force and, in a second position, releasing the torque from the guide wire; and
actuation means arranged in the traverse opening of the elongated body in contact with the clamping body for actuating the clamping body between the first and second positions.

16. The device of claim 15, wherein the through-going bore has a section of enlarged cross-sectional area and the clamping body is provided in that section.

17. The device of claim 15, wherein the clamping body comprises two opposed, elongated parallel wall parts diverging from each other towards the traverse opening to define a gap therebetween for receiving the guide wire.

18. The device of claim 17, wherein the opposed, elongated parallel wall parts are interconnected opposite the traverse opening.

19. The device of claim 15, wherein the clamping body further comprises spring means for forcing the elongated walls of the clamping body towards each other.

20. The device of claim 19, wherein the spring means is a U-shaped spring which engages at least part of the clamping body opposite the traverse opening.

21. The device of claim 19, wherein the spring means is an integrated part of the clamping body.

22. The device of claim 15, wherein the actuation means is an actuation button extending through the traversing opening.

23. The device of claim 22, wherein the actuation button includes wedge means.

24. The device of claim 15, wherein the actuation means is displaceable in at least one of the directions (F,B) substantially along the longitudinal axis of the elongated body and substantially perpendicular (P₁,P₂) to the longitudinal axis (A) of the elongated body.

25. The device of claim 15, wherein the elongated body has a gripping part of reduced diameter.

26. The device of claim 25, wherein the gripping part has an uneven exterior surface.

27. The device of claim 17, wherein at least the opposing faces of the opposed wall parts of the clamping body have a coefficient of friction of at least 0.02.

28. The device of claim 1, wherein at least the opposing faces of the opposed wall parts of the clamping body have a coefficient of friction of at least 0.03.

29. The device of claim 15, in combination with a guide wire.

30. A method of using the medical guide wire torque device of claim 15, which comprises:
positioning a guide wire in the through-going bore of the elongated body; and
activating the actuation means to control the clamping force of the clamping body on the guide wire.

31. The method according to claim 30, which further comprises displacing the torque in the second position of the clamping body.

32. The method according to claim 30, which further comprises rotating the torque fixed on the guide wire in the first position of the clamping body.
